(19) United States
(12) Patent Application Publication
(10) Pub. No.: US 2003/0008301 A1
(43) Pub. Date: Jan. 9, 2003

(54) Association between schizophrenia and a two-marker haplotype near PILB gene

(76) Inventors: Pamela Sklar, Brookline, MA (US); Eric S. Lander, Cambridge, MA (US); Sibylle Schwab, Bonn (DE); Dieter Wildenauer, Bonn (DE)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/061,119

(22) Filed: Feb. 1, 2002

Related U.S. Application Data
(60) Provisional application No. 60/265,910, filed on Feb. 2, 2001.

(51) Int. Cl. C12Q 1/68; C07H 21/04

(52) U.S. Cl. 435/6; 536/23.2

(57) ABSTRACT

Methods for diagnosing and treating neuropsychiatric disorders, especially schizophrenia, and methods for identifying compounds for use in the diagnosis and treatment of neuropsychiatric disorders are disclosed. Also disclosed are novel compounds and pharmaceutical compositions for use in the diagnosis and treatment of neuropsychiatric disorders such as schizophrenia.

### RELATED APPLICATIONS

This application claims priority to U.S. Provisional Application No. 60/265,910 filed Feb. 2, 2001, the contents of which are incorporated by reference in their entirety.

### BACKGROUND OF THE INVENTION

Neuropsychiatric disorders, such as schizophrenia, attention deficit disorders, schizoaffective disorders, bipolar disorders and unipolar disorders, differ from neurological disorders in that anatomical or biochemical pathologies are readily detectable for the latter but not the former. Largely as a result of this difference, drugs which have been used to treat individuals with neuropsychiatric disorders, including lithium salts, valproic acid and carbamazepine, have not been predictably effective in treatment regimens across a variety of patients. Treatment regimens are further complicated by the fact that clinical diagnosis currently relies on clinical observation and subjective reports. Identification of the anatomical or biochemical defects which result in neuropsychiatric disorders is needed in order to effectively distinguish between disorders and to allow the design and administration of effective therapeutics for these disorders.

### SUMMARY OF THE INVENTION

As described herein, it has been discovered that one form of a single nucleotide polymorphism (SNP) near the pilin-like transcription factor PILB gene is negatively correlated with incidence of neuropsychiatric disorders (e.g., schizophrenia). This SNP is 30 kb upstream of PILB. In particular, it has been discovered that the presence of the variant nucleotide (T) at the polymorphic position of SEQ ID NO: 1 (shown in FIG. 1) is correlated with reduced incidence of schizophrenia in the sample population assessed as described herein. That is, it has been determined that there is a variation from random (i.e., that which would be expected by chance) in the transmission of the reference (C) and variant (T) alleles from a parent who is heterozygous for these alleles to an offspring diagnosed with schizophrenia. The variant allele (T) is transmitted less frequently to the schizophrenic offspring than would be expected by chance, while the reference allele (C) is transmitted more frequently than would be expected by chance (p<0.001). Thus, it appears that the variant allele may contribute to protection or reduction in symptomology with respect to schizophrenia. Alternatively, this particular polymorphism may be one of a group of two or more polymorphisms in the area of the PILB gene which contributes to the presence, absence or severity of the neuropsychiatric disorder, e.g., schizophrenia.

Moreover, it has also been discovered that a haplotype containing the variant nucleotides at the polymorphic positions of SEQ ID NOS: 1 and 2 (FIG. 1) is correlated with reduced incidence of schizophrenia in the sample population assessed as described herein. That is, it has been determined that there is a variation from random (i.e., that which would be expected by chance) in the co-transmission of the reference (C and A, respectively) and variant (T and G, respectively) forms from a parent who is heterozygous to an offspring diagnosed with schizophrenia. The variant haplotype (comprising the T and G nucleotides, respectively, at the polymorphic positions) is transmitted less frequently to the schizophrenic offspring than would be expected by chance, while the reference haplotype (comprising the C and A, respectively, nucleotides at the polymorphic positions) is transmitted more frequently than would be expected by chance (p<0.01). Thus, it appears that the variant haplotype may contribute to protection or reduction in symptomology with respect to schizophrenia.

Accordingly, the invention relates to methods for diagnosing and treating neuropsychiatric disorders, especially schizophrenia, and to methods for identifying compounds for use in the diagnosis and treatment of neuropsychiatric disorders. The invention further relates to novel compounds and pharmaceutical compositions for use in the diagnosis and treatment of neuropsychiatric disorders. In a preferred embodiment, the neuropsychiatric disorder is schizophrenia.

In one embodiment, the invention relates to a method for predicting the likelihood that an individual will have a neuropsychiatric disorder (or aiding in the diagnosis of a neuropsychiatric disorder), e.g., schizophrenia, comprising the steps of obtaining a DNA sample from an individual to be assessed and determining the nucleotide present at the polymorphic position of SEQ ID NO: 1. The presence of a “T” (the variant nucleotide) at this position indicates that the individual has a lower likelihood of having a neuropsychiatric disorder than an individual having a “C” at that position, or a greater likelihood of having less severe symptomology. In a preferred embodiment, the neuropsychiatric disorder is schizophrenia. In a particular embodiment, the individual is an individual at risk for development of schizophrenia.

In one embodiment, the invention relates to a method for predicting the likelihood that an individual will have a neuropsychiatric disorder (or aiding in the diagnosis of a neuropsychiatric disorder), e.g., schizophrenia, comprising the steps of obtaining a DNA sample from an individual to be assessed and determining the nucleotide present at the polymorphic position of SEQ ID NO: 2. The presence of a “G” (the variant nucleotide) at this position indicates that the individual has a lower likelihood of having a neuropsychiatric disorder than an individual having an “A” at that position, or a greater likelihood of having less severe symptomology. In a preferred embodiment, the neuropsychiatric disorder is schizophrenia. In a particular embodiment, the individual is an individual at risk for development of schizophrenia.

In another embodiment, the invention relates to a method for predicting the likelihood that an individual will have a neuropsychiatric disorder (or aiding in the diagnosis of a neuropsychiatric disorder), e.g., schizophrenia, comprising the steps of obtaining a DNA sample from an individual to be assessed and determining the nucleotide present at the polymorphic position of SEQ ID NO: 1 and the nucleotide present at the polymorphic position of SEQ ID NO: 2. The presence of a “T” and a “G”, respectively, (the variant nucleotides; collectively, the variant haplotype)) at these positions indicates that the individual has a lower likelihood of having a neuropsychiatric disorder than an individual having a “C” and an “A” at these positions, or a greater likelihood of having less severe symptomology. In a preferred embodiment, the neuropsychiatric disorder is schizophrenia. In a particular embodiment, the individual is an individual at risk for development of bipolar disorder.

In another embodiment, the invention relates to pharmaceutical compositions comprising a variant gene product of the invention for use in the treatment of neuropsychiatric disorders. The invention also relates to the use of a nucleic acid molecule encoding a variant gene product of the invention for use in the treatment of neuropsychiatric disorders. In a particular embodiment the neuropsychiatric disorder is schizophrenia.

The invention further relates to an oligonucleotide microarray having immobilized thereon a plurality of oligonucleotide probes wherein at least one of said probes is specific for the variant nucleotide at the polymorphic position of SEQ ID NO: 1 or SEQ ID NO: 2. In another embodiment, the invention relates to an oligonucleotide microarray having immobilized thereon a plurality of oligonucleotide probes wherein at least one of said probes is specific for the variant nucleotide at the polymorphic position of SEQ ID NO: 1 and wherein at least one of said probes is specific for the variant nucleotide at the polymorphic position of SEQ ID NO: 2.

The invention further relates to an oligonucleotide microarray having immobilized thereon a plurality of oligonucleotide probes wherein at least one of said probes is specific for the reference nucleotide at the polymorphic position of SEQ ID NO: 1 or SEQ ID NO: 2. In another embodiment, the invention relates to an oligonucleotide microarray having immobilized thereon a plurality of oligonucleotide probes wherein at least one of said probes is specific for the reference nucleotide at the polymorphic position of SEQ ID NO: 1 and wherein at least one of said probes is specific for the reference nucleotide at the polymorphic position of SEQ ID NO: 2.

### BRIEF DESCRIPTION OF THE DRAWINGS

FIG. 1 shows the polymorphic sites and surrounding nucleic acid sequences of the TSC0615653 and TSC0426297 SNPs (SEQ ID NOS: 1 and 2, respectively).

FIGS. 2A-2I show the nucleotide sequence (SEQ ID NO: 4) of the region of chromosome 10 (25076536-25101913) which contains the PILB gene.

FIG. 3 shows a schematic of the region of chromosome 10 containing the PILB gene.

FIGS. 4A-4B is a printout from NCBI for the PILB gene sequence (SEQ ID NO: 5) and protein sequence (SEQ ID NO: 3).

### DETAILED DESCRIPTION OF THE INVENTION

As used herein, polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphic locus may be as small as one base pair, in which case it is referred to as a single nucleotide polymorphism.

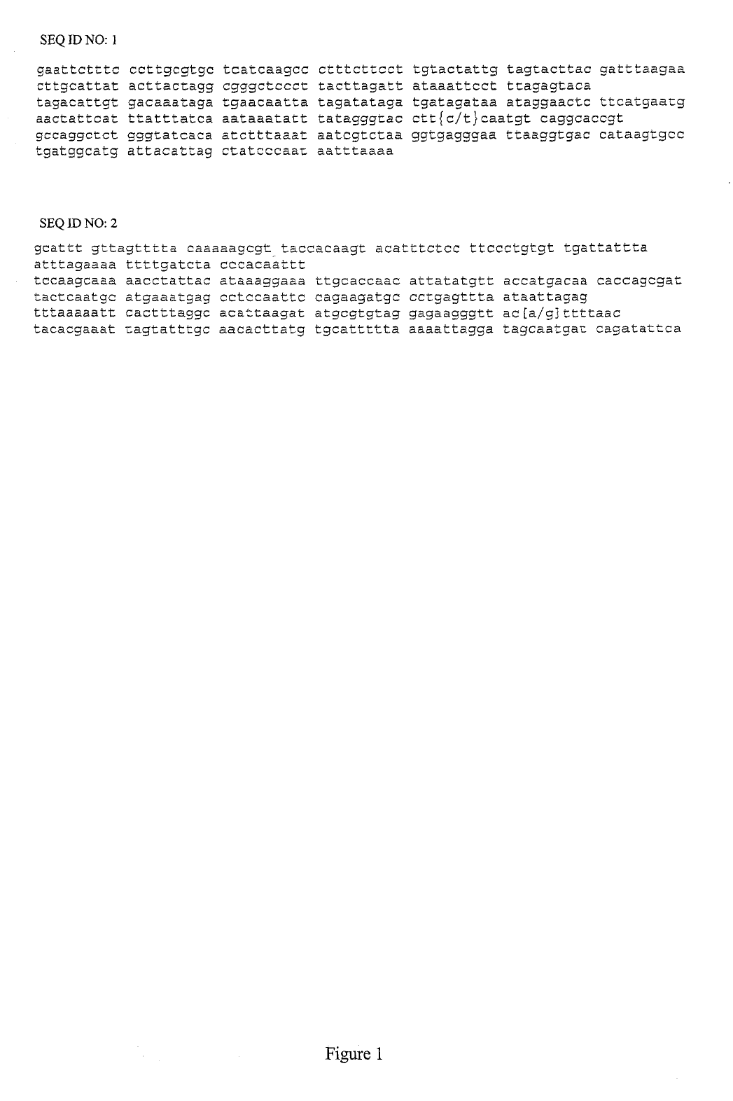

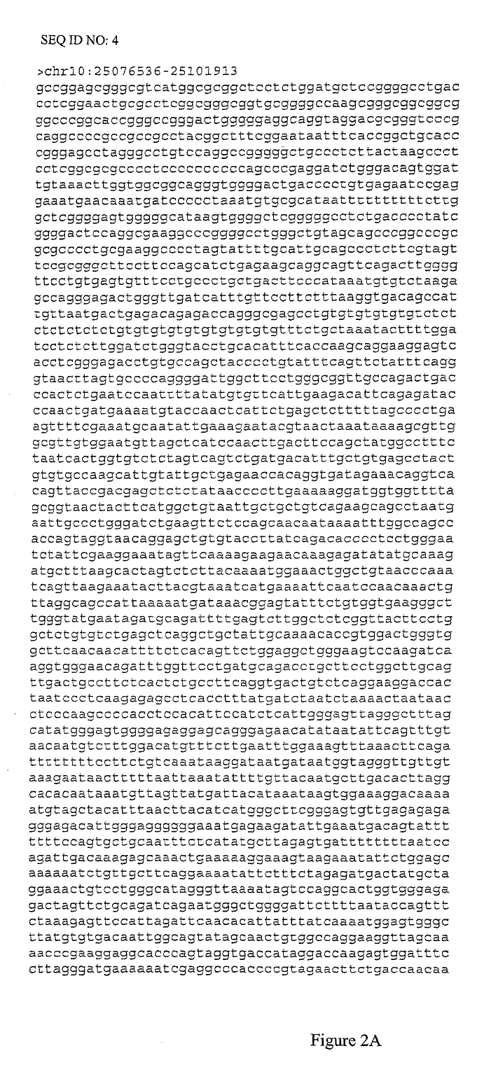

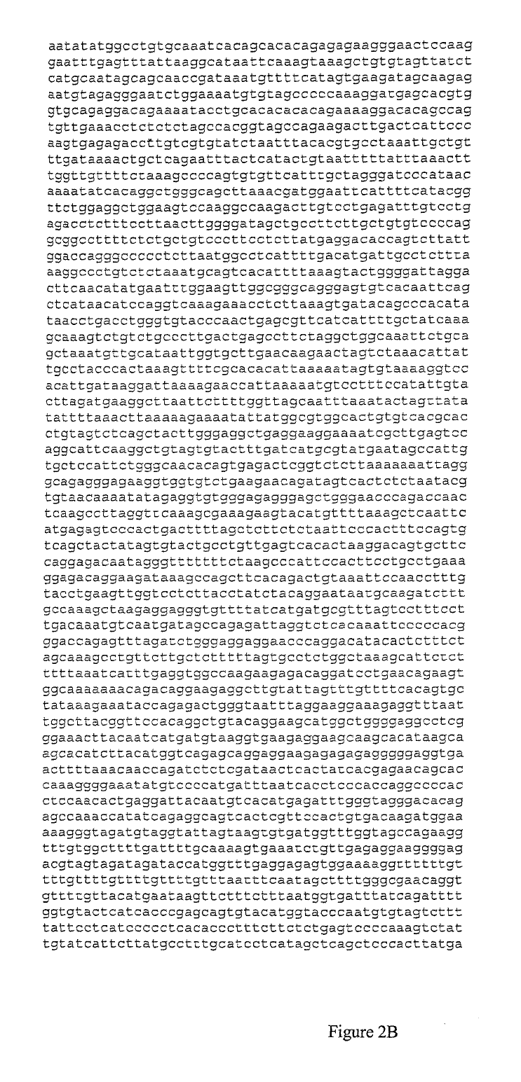

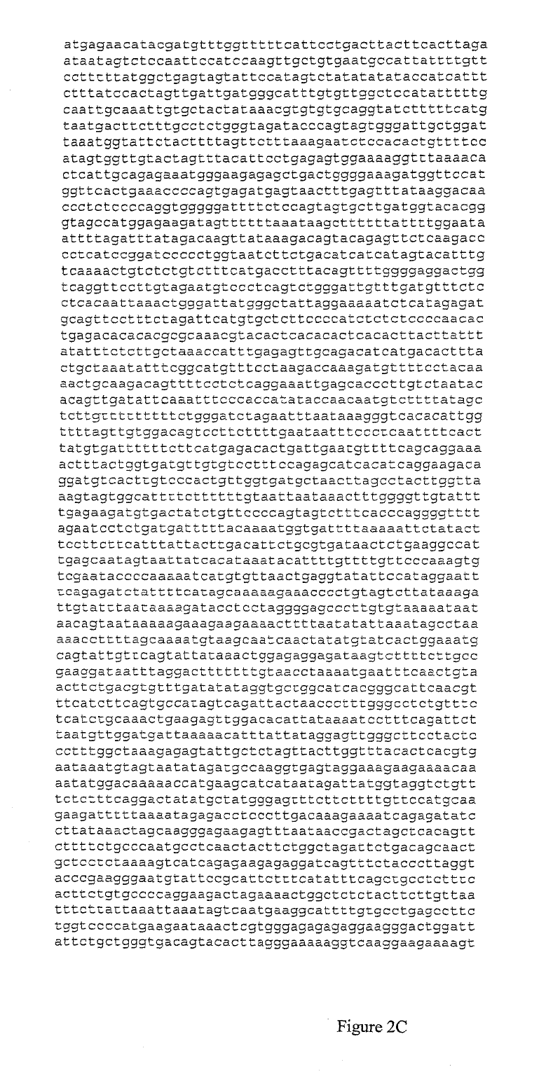

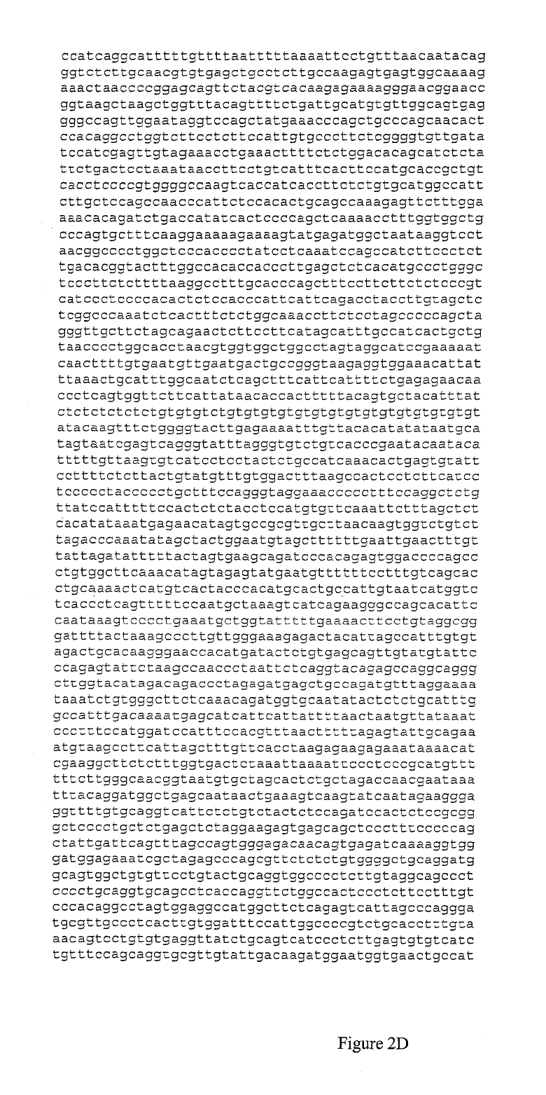

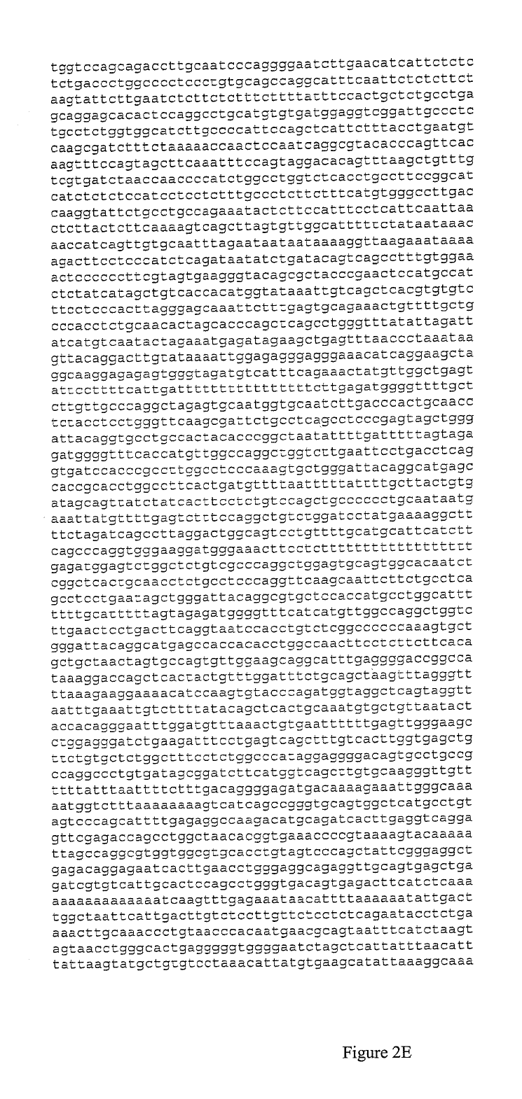

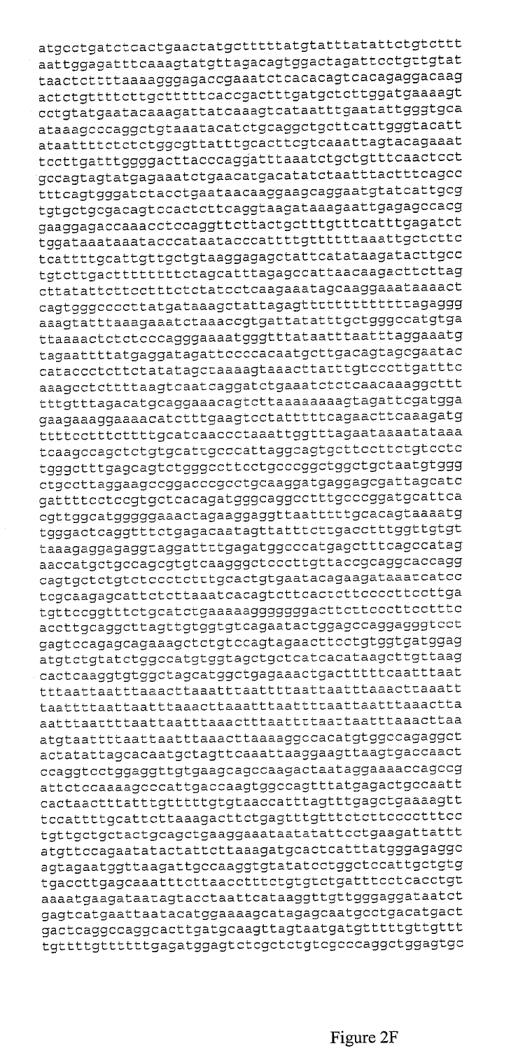

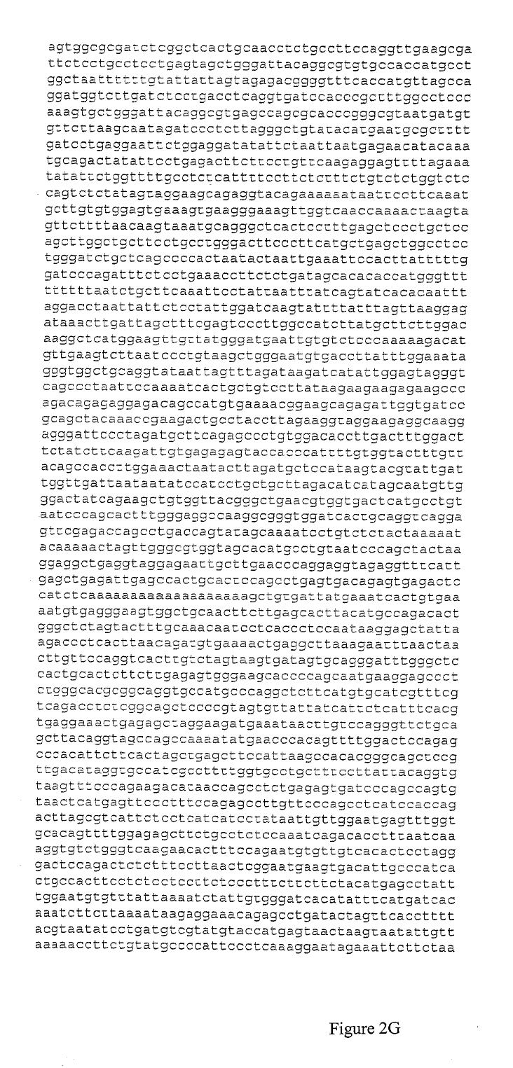

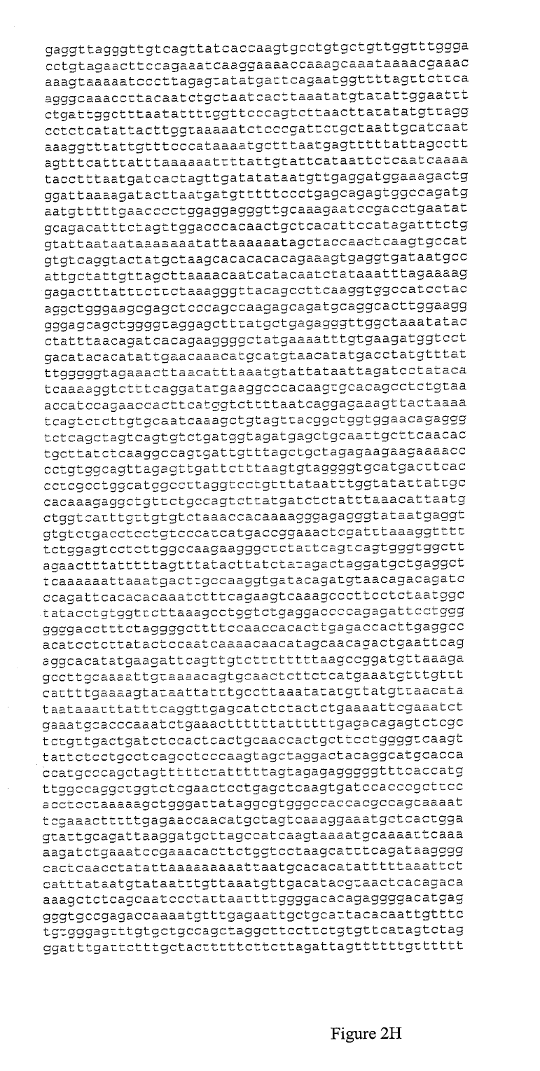

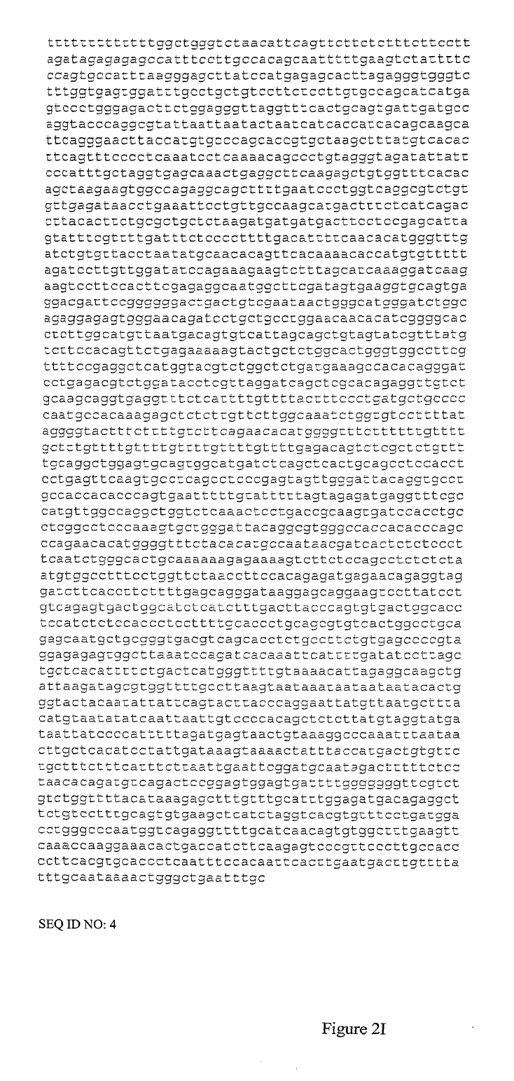

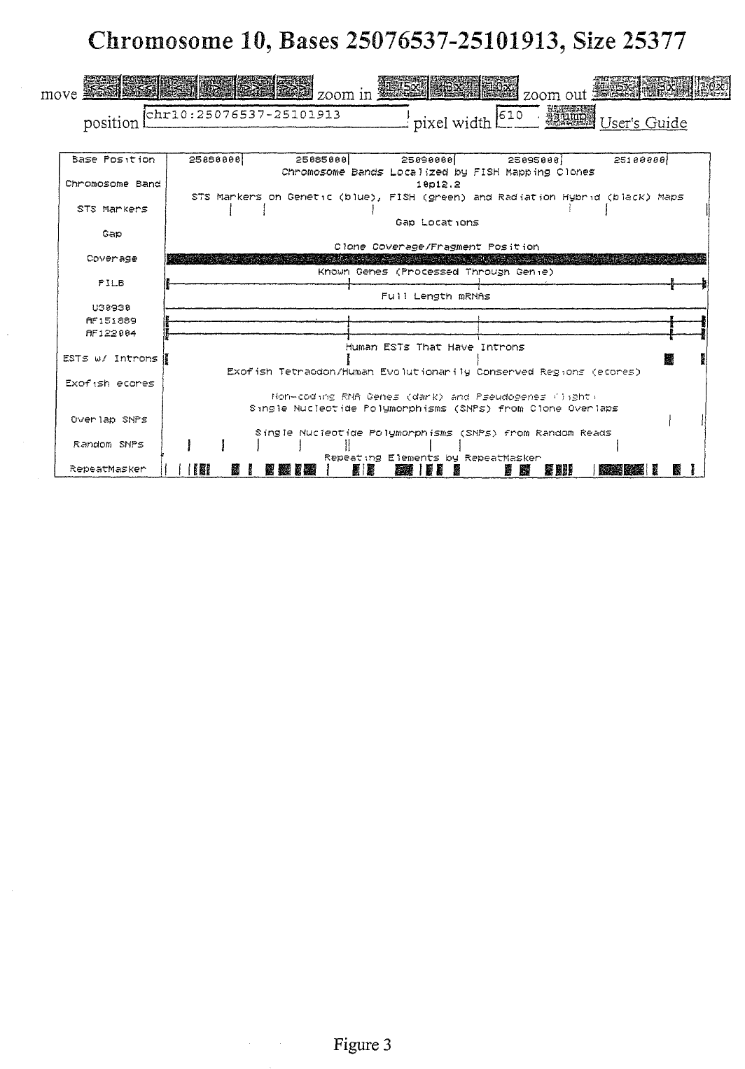

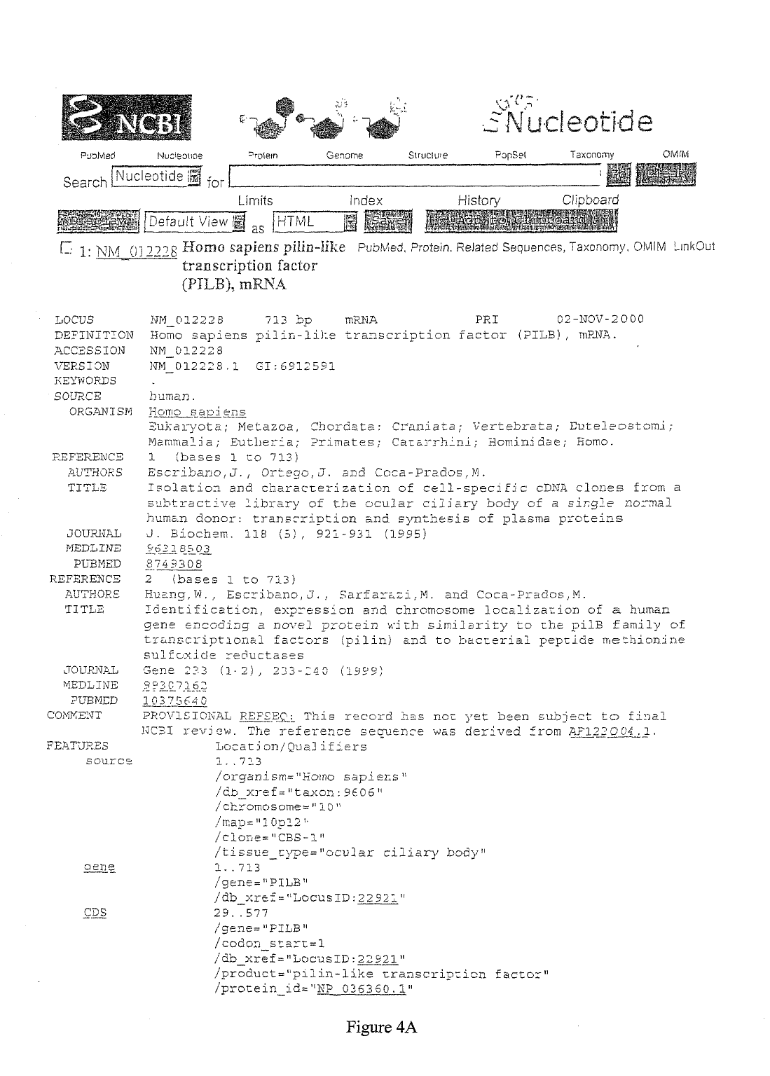

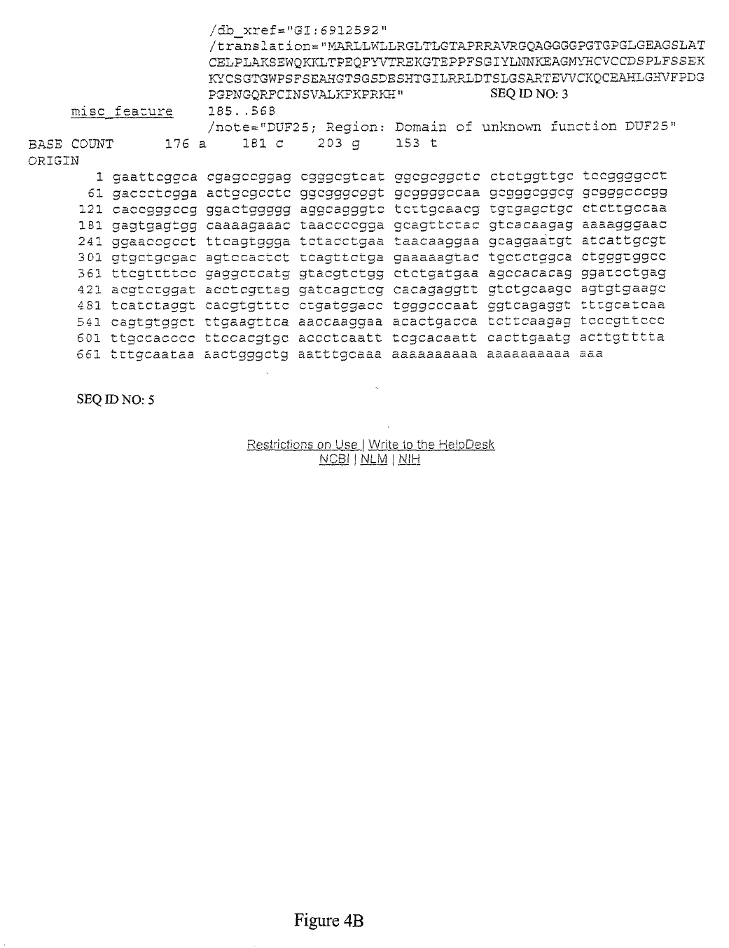

As described herein, it has been discovered that one form of a single nucleotide polymorphism (SNP) near the pilin-like transcription factor PILB gene is negatively correlated with incidence of neuropsychiatric disorders (e.g., schizophrenia). This SNP is 30 kb upstream of PILB. In particular, it has been discovered that the presence of the variant nucleotide (T) at the polymorphic position of SEQ ID NO: 1 (FIG. 1) is correlated with reduced incidence of schizophrenia in the sample population assessed as described herein. That is, it has been determined that there is a variation from random (i.e., that which would be expected by chance) in the transmission of the reference (C) and variant (T) alleles from a parent who is heterozygous for these alleles to an offspring diagnosed with schizophrenia. The variant allele (T) is transmitted less frequently to the schizophrenic offspring than would be expected by chance, while the reference allele (C) is transmitted more frequently than would be expected by chance (p<0.001). Thus, it appears that the variant allele may contribute to protection or reduction in symptomology with respect to schizophrenia. Alternatively, this particular polymorphism may be one of a group of two or more polymorphisms in the area of the PILB gene which contributes to the presence, absence or severity of the neuropsychiatric disorder, e.g., schizophrenia.

Moreover, it has also been discovered that a haplotype containing the variant nucleotides at the polymorphic positions of SEQ ID NOS: 1 and 2 (FIG. 1) is correlated with reduced incidence of schizophrenia in the sample population assessed as described herein. That is, it has been determined that there is a variation from random (i.e., that which would be expected by chance) in the co-transmission of the reference (C and A, respectively) and variant (T and G, respectively) forms from a parent who is heterozygous to an offspring diagnosed with schizophrenia. The variant haplotype (comprising the T and G nucleotides, respectively, at the polymorphic positions) is transmitted less frequently to the schizophrenic offspring than would be expected by chance, while the reference haplotype (comprising the C and A, respectively, nucleotides at the polymorphic positions) is transmitted more frequently than would be expected by chance (p<0.01). Thus, it appears that the variant haplotype may contribute to protection or reduction in symptomology with respect to schizophrenia.

Thus, the invention relates to a method for predicting the likelihood that an individual will have a neuropsychiatric disorder, or for aiding in the diagnosis of a neuropsychiatric disorder, e.g., schizophrenia, or for predicting a greater likelihood of an individual's having reduced symptomology associated with a neuropsychiatric disorder, e.g., schizophrenia, comprising the steps of obtaining a DNA sample from an individual to be assessed and determining the nucleotide present at the polymorphic position of SEQ ID NO: 1. The presence of a “T” (the variant nucleotide) at this position indicates that the individual has a lower likelihood of having a neuropsychiatric disorder, or a greater likelihood of having reduced symptomology associated with a neuropsychiatric disorder, than if that individual had the reference nucleotide at that position. Conversely, the presence of a “C” (the reference nucleotide) at this position indicates that the individual has a greater likelihood of having a neuropsychiatric disorder, or a likelihood of having increased symptomology associated with a neuropsychiatric disorder, than if that individual had the variant nucleotide at that position. In a preferred embodiment, the neuropsychiatric disorder is schizophrenia. In a particular embodiment, the individual is an individual at risk for development of schizophrenia. In another embodiment the individual exhibits clinical symptomology associated with schizophrenia. In one embodiment, the individual has been clinically diagnosed as having schizophrenia.

Thus, the invention relates to a method for predicting the likelihood that an individual will have a neuropsychiatric disorder, or for aiding in the diagnosis of a neuropsychiatric disorder, e.g., schizophrenia, or for predicting a greater likelihood of an individual's having reduced symptomology associated with a neuropsychiatric disorder, e.g., schizophrenia, comprising the steps of obtaining a DNA sample from an individual to be assessed and determining the nucleotide present at the polymorphic position of SEQ ID NO: 2. The presence of a “G” (the variant nucleotide) at this position indicates that the individual has a lower likelihood of having a neuropsychiatric disorder, or a greater likelihood of having reduced symptomology associated with a neuropsychiatric disorder, than if that individual had the reference nucleotide at that position. Conversely, the presence of an “A” (the reference nucleotide) at this position indicates that the individual has a greater likelihood of having a neuropsychiatric disorder, or a likelihood of having increased symptomology associated with a neuropsychiatric disorder, than if that individual had the variant nucleotide at that position. In a preferred embodiment, the neuropsychiatric disorder is schizophrenia. In a particular embodiment, the individual is an individual at risk for development of schizophrenia. In another embodiment the individual exhibits clinical symptomology associated with schizophrenia. In one embodiment, the individual has been clinically diagnosed as having schizophrenia.

The invention also relates to a method for predicting the likelihood that an individual will have a neuropsychiatric disorder, or for aiding in the diagnosis of a neuropsychiatric disorder, e.g., schizophrenia, or for predicting a greater likelihood of an individual's having reduced symptomology associated with a neuropsychiatric disorder, e.g., schizophrenia, comprising the steps of obtaining a DNA sample from an individual to be assessed and determining the nucleotide present at the polymorphic position of SEQ ID NO: 1 and the polymorphic position of SEQ ID NO: 2. The presence of the variant haplotype (i.e., both a T and a G, respectively) indicates that the individual has a lower likelihood of having a neuropsychiatric disorder, or a greater likelihood of having reduced symptomology associated with a neuropsychiatric disorder, than if that individual had the reference haplotype. Conversely, the presence of the reference haplotype (i.e., both a C and an A, respectively) indicates that the individual has a greater likelihood of having a neuropsychiatric disorder, or a likelihood of having increased symptomology associated with a neuropsychiatric disorder, than if that individual had the variant haplotype. In a preferred embodiment, the neuropsychiatric disorder is schizophrenia. In a particular embodiment, the individual is an individual at risk for development of schizophrenia. In another embodiment the individual exhibits clinical symptomology associated with schizophrenia. In one embodiment, the individual has been clinically diagnosed as having schizophrenia.

The genetic material to be assessed can be obtained from any nucleated cell from the individual. For assay of genomic DNA, virtually any biological sample (other than pure red blood cells) is suitable. For example, convenient tissue samples include whole blood, semen, saliva, tears, urine, fecal material, sweat, skin and hair. For assay of cDNA or mRNA, the tissue sample must be obtained from an organ in which the target nucleic acid is expressed.

Many of the methods described herein require amplification of DNA from target samples. This can be accomplished by, e.g., PCR. See generally PCR Technology: Principles and Applications for DNA Amplification (ed. H. A. Erlich, Freeman Press, NY, N.Y., 1992); PCR Protocols: A Guide to Methods and Applications (eds. Innis, et al., Academic Press, San Diego, Calif., 1990); Mattila et al., Nucleic Acids Res. 19, 4967 (1991); Eckert et al., PCR Methods and Applications 1, 17 (1991); PCR (eds. McPherson et al., IRL Press, Oxford); and U.S. Pat. No. 4,683,202.

Other suitable amplification methods include the ligase chain reaction (LCR) (see Wu and Wallace, Genomics 4, 560 (1989), Landegren et al., Science 241, 1077 (1988), transcription amplification (Kwoh et al., Proc. Natl. Acad. Sci. USA 86, 1173 (1989)), and self-sustained sequence replication (Guatelli et al., Proc. Nat. Acad. Sci. USA, 87, 1874 (1990)) and nucleic acid based sequence amplification (NASBA). The latter two amplification methods involve isothermal reactions based on isothermal transcription, which produce both single stranded RNA (ssRNA) and double stranded DNA (dsDNA) as the amplification products in a ratio of about 30 or 100 to 1, respectively.

The nucleotide which occupies the polymorphic site of interest can be identified by a variety methods, such as Southern analysis of genomic DNA; direct mutation analysis by restriction enzyme digestion; Northern analysis of RNA; denaturing high pressure liquid chromatography (DHPLC); gene isolation and sequencing; hybridization of an allele-specific oligonucleotide with amplified gene products; single base extension (SBE); or protein analysis. In a preferred embodiment, determination of the allelic form of a gene is carried out using SBE-FRET methods as described in the examples, or using chip-based oligonucleotide arrays. A sampling of suitable procedures are discussed below in turn.

1. Allele-Specific Probes

The design and use of allele-specific probes for analyzing polymorphisms is described by e.g., Saiki et al., Nature 324, 163-166 (1986); Dattagupta, EP 235,726, Saiki, WO 89/11548. Allele-specific probes can be designed that hybridize to a segment of target DNA from one individual but do not hybridize to the corresponding segment from another individual due to the presence of different polymorphic forms in the respective segments from the two individuals. Hybridization conditions should be sufficiently stringent that there is a significant difference in hybridization intensity between alleles, and preferably an essentially binary response, whereby a probe hybridizes to only one of the alleles. Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C., or equivalent conditions, are suitable for allele-specific probe hybridizations. Equivalent conditions can be determined by varying one or more of the parameters given as an example, as known in the art, while maintaining a similar degree of identity or similarity between the target nucleotide sequence and the primer or probe used.

Some probes are designed to hybridize to a segment of target DNA such that the polymorphic site aligns with a central position (e.g., in a 15-mer at the 7 position; in a 16-mer, at either the 8 or 9 position) of the probe. This design of probe achieves good discrimination in hybridization between different allelic forms.

Allele-specific probes are often used in pairs, one member of a pair showing a perfect match to a reference form of a target sequence and the other member showing a perfect match to a variant form. Several pairs of probes can then be immobilized on the same support for simultaneous analysis of multiple polymorphisms within the same target sequence.

2. Tiling Arrays

The polymorphisms can also be assessed by hybridization to nucleic acid arrays, some examples of which are described in WO 95/11995. WO 95/11995 also describes subarrays that are optimized for detection of a variant form of a precharacterized polymorphism. Such a subarray contains probes designed to be complementary to a second reference sequence, which is an allelic variant of the first reference sequence. The second group of probes is designed by the same principles, except that the probes exhibit complementarity to the second reference sequence. The inclusion of a second group (or further groups) can be particularly useful for analyzing short subsequences of the primary reference sequence in which multiple mutations are expected to occur within a short distance commensurate with the length of the probes (e.g., two or more mutations within 9 to 21 bases).

3. Allele-Specific Primers

An allele-specific primer hybridizes to a site on target DNA overlapping a polymorphism and only primes amplification of an allelic form to which the primer exhibits perfect complementarity. See Gibbs, Nucleic Acid Res. 17, 2427-2448 (1989). This primer is used in conjunction with a second primer which hybridizes at a distal site. Amplification proceeds from the two primers, resulting in a detectable product which indicates the particular allelic form is present. A control is usually performed with a second pair of primers, one of which shows a single base mismatch at the polymorphic site and the other of which exhibits perfect complementarity to a distal site. The single-base mismatch prevents amplification and no detectable product is formed. The method works best when the mismatch is included in the 3′-most position of the oligonucleotide aligned with the polymorphism because this position is most destabilizing to elongation from the primer (see, e.g., WO 93/22456).

4. Direct-Sequencing

The direct analysis of the sequence of polymorphisms of the present invention can be accomplished using either the dideoxy chain termination method or the Maxam-Gilbert method (see Sambrook et al., Molecular Cloning, A Laboratory Manual (2nd Ed., CSHP, New York 1989); Zyskind et al., Recombinant DNA Laboratory Manual, (Acad. Press, 1988)).

5. Denaturing Gradient Gel Electrophoresis

Amplification products generated using the polymerase chain reaction can be analyzed by the use of denaturing gradient gel electrophoresis. Different alleles can be identified based on the different sequence-dependent melting properties and electrophoretic migration of DNA in solution. Erlich, ed., PCR Technology, Principles and Applications for DNA Amplification, (W. H. Freeman and Co, New York, 1992), Chapter 7.

6. Single-Strand Conformation Polymorphism Analysis

Alleles of target sequences can be differentiated using single-strand conformation polymorphism analysis, which identifies base differences by alteration in electrophoretic migration of single stranded PCR products, as described in Orita et al., Proc. Nat. Acad. Sci. 86, 2766-2770 (1989). Amplified PCR products can be generated as described above, and heated or otherwise denatured, to form single stranded amplification products. Single-stranded nucleic acids may refold or form secondary structures which are partially dependent on the base sequence. The different electrophoretic mobilities of single-stranded amplification products can be related to base-sequence differences between alleles of target sequences.

7. Single-Base Extension

An alternative method for identifying and analyzing polymorphisms is based on single-base extension (SBE) of a fluorescently-labeled primer coupled with fluorescence resonance energy transfer (FRET) between the label of the added base and the label of the primer. Typically, the method, such as that described by Chen et al., (PNAS 94:10756-61 (1997), incorporated herein by reference) uses a locus-specific oligonucleotide primer labeled on the 5′ terminus with 5-carboxyfluorescein (FAM). This labeled primer is designed so that the 3′ end is immediately adjacent to the polymorphic site of interest. The labeled primer is hybridized to the locus, and single base extension of the labeled primer is performed with fluorescently labeled dideoxyribonucleotides (ddNTPs) in dye-terminator sequencing fashion, except that no deoxyribonucleotides are present. An increase in fluorescence of the added ddNTP in response to excitation at the wavelength of the labeled primer is used to infer the identity of the added nucleotide.

The polymorphisms of the invention may contribute to the protection of an individual against schizophrenia in different ways. The polymorphisms may exert phenotypic effects indirectly via influence on replication, transcription, and translation. More than one phenotypic trait may be affected. For example, other neuropsychiatric disorders, such as bipolar disorder, variants of schizoaffective disorder, recurrent unipolar depression and hypomania (bipolar II disorder), may also be affected by the polymorphisms described herein. Additionally, the described polymorphisms may predispose an individual to a distinct mutation that is causally related to a certain phenotype, such as susceptibility or resistance to schizophrenia. The discovery of the polymorphisms of the invention and, in particular, their correlation with schizophrenia, facilitates biochemical analysis of the variant and the development of assays to characterize the variant and to screen for pharmaceuticals that interact directly with one or another form of the protein.

Alternatively, this particular polymorphism may be one of a group of two or more polymorphisms in this area which contributes to the presence, absence or severity of the neuropsychiatric disorder, e.g., schizophrenia. An assessment of other polymorphisms within this area can be undertaken, and the separate and combined effects of these polymorphisms on the neuropsychiatric disorder phenotype can be assessed.

Correlation between a particular phenotype, e.g., the schizophrenic phenotype, and the presence or absence of a particular allele is performed for a population of individuals who have been tested for the presence or absence of the phenotype. Correlation can be performed by standard statistical methods such as a Chi-squared test and statistically significant correlations between polymorphic form(s) and phenotypic characteristics are noted.

This correlation can be exploited in several ways. In the case of a strong correlation between a particular polymorphic form, e.g., the reference allele or haplotype, and a disease for which treatment is available, e.g., schizophrenia, detection of the polymorphic form in an individual may justify immediate administration of treatment, or at least the institution of regular monitoring of the individual. Detection of a polymorphic form correlated with a disorder in a couple contemplating a family may also be valuable to the couple in their reproductive decisions. For example, the female partner might elect to undergo in vitro fertilization to avoid the possibility of transmitting such a polymorphism from her husband to her offspring. In the case of a weaker, but still statistically significant correlation between a polymorphic form and a particular disorder, immediate therapeutic intervention or monitoring may not be justified. Nevertheless, the individual can be motivated to begin simple life-style changes (e.g., therapy or counseling) that can be accomplished at little cost to the individual but confer potential benefits in reducing the risk of conditions to which the individual may have increased susceptibility by virtue of the particular allele. Furthermore, identification of a polymorphic form correlated with enhanced receptiveness to one of several treatment regimes for a disorder indicates that this treatment regime should be followed for the individual in question.

Furthermore, it may be possible to identify a physical linkage between a genetic locus associated with a trait of interest (e.g., schizophrenia) and polymorphic markers that are not associated with the trait, but are in physical proximity with the genetic locus responsible for the trait and co-segregate with it. Such analysis is useful for mapping a genetic locus associated with a phenotypic trait to a chromosomal position, and thereby cloning gene(s) responsible for the trait. See Lander et al., Proc. Natl. Acad. Sci. (USA) 83, 7353-7357 (1986); Lander et al., Proc. Natl. Acad. Sci. (USA) 84, 2363-2367 (1987); Donis-Keller et al., Cell 51, 319-337 (1987); Lander et al., Genetics 121, 185-199 (1989)). Genes localized by linkage can be cloned by a process known as directional cloning. See Wainwright, Med. J. Australia 159, 170-174 (1993); Collins, Nature Genetics 1, 3-6 (1992).

Linkage studies are typically performed on members of a family. Available members of the family are characterized for the presence or absence of a phenotypic trait and for a set of polymorphic markers. The distribution of polymorphic markers in an informative meiosis is then analyzed to determine which polymorphic markers co-segregate with a phenotypic trait. See, e.g., Kerem et al., Science 245, 1073-1080 (1989); Monaco et al., Nature 316, 842 (1985); Yamoka et al., Neurology 40, 222-226 (1990); Rossiter et al., FASEB Journal 5, 21-27 (1991).

Linkage is analyzed by calculation of LOD (log of the odds) values. A lod value is the relative likelihood of obtaining observed segregation data for a marker and a genetic locus when the two are located at a recombination fraction θ, versus the situation in which the two are not linked, and thus segregating independently (Thompson & Thompson, Genetics in Medicine (5th ed, W. B. Saunders Company, Philadelphia, 1991); Strachan, “Mapping the human genome” in The Human Genome (BIOS Scientific Publishers Ltd, Oxford), Chapter 4). A series of likelihood ratios are calculated at various recombination fractions (θ), ranging from θ=0.0 (coincident loci) to θ=0.50 (unlinked). Thus, the likelihood at a given value of θ is: probability of data if loci linked at θ to probability of data if loci unlinked. The computed likelihoods are usually expressed as the log₁₀ of this ratio (i.e., a lod score). For example, a lod score of 3 indicates 1000:1 odds against an apparent observed linkage being a coincidence. The use of logarithms allows data collected from different families to be combined by simple addition. Computer programs are available for the calculation of lod scores for differing values of θ (e.g., LIPED, MLINK (Lathrop, Proc. Nat. Acad. Sci. (USA) 81, 3443-3446 (1984)). For any particular lod score, a recombination fraction may be determined from mathematical tables. See Smith et al., Mathematical tables for research workers in human genetics (Churchill, London, 1961); Smith, Ann. Hum. Genet. 32, 127-150 (1968). The value of θ at which the lod score is the highest is considered to be the best estimate of the recombination fraction.

Positive lod score values suggest that the two loci are linked, whereas negative values suggest that linkage is less likely (at that value of θ) than the possibility that the two loci are unlinked. By convention, a combined lod score of +3 or greater (equivalent to greater than 1000:1 odds in favor of linkage) is considered definitive evidence that two loci are linked. Similarly, by convention, a negative lod score of −2 or less is taken as definitive evidence against linkage of the two loci being compared. Negative linkage data are useful in excluding a chromosome or a segment thereof from consideration. The search focuses on the remaining non-excluded chromosomal locations.

The invention further pertains to compositions, e.g., vectors, comprising a nucleotide sequence encoding the variant gene product. For example, variant genes can be expressed in an expression vector in which a variant gene is operably linked to a native or other promoter. Usually, the promoter is a eukaryotic promoter for expression in a mammalian cell. The transcription regulation sequences typically include a heterologous promoter and optionally an enhancer which is recognized by the host. The selection of an appropriate promoter, for example trp, lac, phage promoters, glycolytic enzyme promoters and tRNA promoters, depends on the host selected. Commercially available expression vectors can be used. Vectors can include host-recognized replication systems, amplifiable genes, selectable markers, host sequences useful for insertion into the host genome, and the like.

The means of introducing the expression construct into a host cell varies depending upon the particular construction and the target host. Suitable means include fusion, conjugation, transfection, transduction, electroporation or injection, as described in Sambrook, supra. A wide variety of host cells can be employed for expression of the variant gene, both prokaryotic and eukaryotic. Suitable host cells include bacteria such as E. coli, yeast, filamentous fungi, insect cells, mammalian cells, typically immortalized, e.g., mouse, CHO, human and monkey cell lines and derivatives thereof. Preferred host cells are able to process the variant gene product to produce an appropriate mature polypeptide. Processing includes glycosylation, ubiquitination, disulfide bond formation, general post-translational modification, and the like.

It is also contemplated that cells can be engineered to express the variant allele of the invention by gene therapy methods. For example, DNA encoding the variant gene product, or an active fragment or derivative thereof, can be introduced into an expression vector, such as a viral vector, and the vector can be introduced into appropriate cells in an animal. In such a method, the cell population can be engineered to inducibly or constitutively express active variant gene product. In a preferred embodiment, the vector is delivered to the bone marrow, for example as described in Corey et al. (Science 244:1275-1281 (1989)).

The invention further provides transgenic nonhuman animals capable of expressing an exogenous variant gene and/or having one or both alleles of an endogenous gene inactivated. Expression of an exogenous variant gene is usually achieved by operably linking the gene to a promoter and optionally an enhancer, and microinjecting the construct into a zygote. See Hogan et al., “Manipulating the Mouse Embryo, A Laboratory Manual,” Cold Spring Harbor Laboratory. Inactivation of endogenous variant genes can be achieved by forming a transgene in which a cloned variant gene is inactivated by insertion of a positive selection marker. See Capecchi, Science 244, 1288-1292 (1989). The transgene is then introduced into an embryonic stem cell, where it undergoes homologous recombination with an endogenous variant gene. Mice and other rodents are preferred animals. Such animals provide useful drug screening systems.

The invention further relates to an oligonucleotide microarray having immobilized thereon a plurality of oligonucleotide probes wherein at least one of said probes is specific for the variant nucleotide at the polymorphic position of SEQ ID NO: 1 or SEQ ID NO: 2. In another embodiment, the invention relates to an oligonucleotide microarray having immobilized thereon a plurality of oligonucleotide probes wherein at least one of said probes is specific for the variant nucleotide at the polymorphic position of SEQ ID NO: 1 and wherein at least one of said probes is specific for the variant nucleotide at the polymorphic position of SEQ ID NO: 2.

The invention further relates to an oligonucleotide microarray having immobilized thereon a plurality of oligonucleotide probes wherein at least one of said probes is specific for the reference nucleotide at the polymorphic position of SEQ ID NO: 1 or SEQ ID NO: 2. In another embodiment, the invention relates to an oligonucleotide microarray having immobilized thereon a plurality of oligonucleotide probes wherein at least one of said probes is specific for the reference nucleotide at the polymorphic position of SEQ ID NO: 1 and wherein at least one of said probes is specific for the reference nucleotide at the polymorphic position of SEQ ID NO: 2. The nucleic acid sequence surrounding the polymorphic positions of SEQ ID NO: 1 and SEQ ID NO: 2 can be used to design suitable oligonucleotide probes, and the preparation of such oligonucleotide microarrays is well known in the art.

The invention also encompasses kits for detecting the presence of proteins or nucleic acid molecules of the invention in a biological sample. For example, the kit can comprise a labeled compound or agent (e.g., nucleic acid molecule, antibody, etc.) capable of detecting protein, DNA or mRNA in a biological sample; means for determining the amount of in the sample; and means for comparing the amount of in the sample with a standard. The compound or agent can be packaged in a suitable container. The kit can further comprise instructions for using the kit to detect protein or nucleic acid. In a preferred embodiment the labeled compound or agent detects either the alternate or reference form of the protein, DNA or mRNA, but not both.

The teachings of the references cited herein are incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

What is claimed is:

1. A method for predicting the likelihood that an individual will have a neuropsychiatric disorder, comprising the steps of:
a) obtaining a DNA sample from an individual to be assessed; and
b) determining the nucleotide present at the polymorphic position in SEQ ID NO: 1,
wherein the presence of an “C” at this position indicates that the individual has an increased likelihood of having a neuropsychiatric disorder than an individual having a “T” at that position.

2. A method according to claim 1, wherein the neuropsychiatric disorder is schizophrenia.

3. A method according to claim 1, wherein the individual is an individual at risk for development of schizophrenia.

4. A method for predicting the likelihood that an individual will have a neuropsychiatric disorder, comprising the steps of:
a) obtaining a DNA sample from an individual to be assessed; and
b) determining the nucleotide present at the polymorphic position in SEQ ID NO: 2,
wherein the presence of an “A” at this position indicates that the individual has an increased likelihood of having a neuropsychiatric disorder than an individual having a “G” at that position.

5. A method according to claim 4, wherein the neuropsychiatric disorder is schizophrenia.

6. A method according to claim 4, wherein the individual is an individual at risk for development of schizophrenia.

7. A method for predicting the likelihood that an individual will have reduced symptomology associated with a neuropsychiatric disorder, comprising the steps of:
a) obtaining a DNA sample from an individual to be assessed; and
b) determining the nucleotide present at the polymorphic position in SEQ ID NO: 1,
wherein the presence of an “T” at this position indicates that the individual has a greater likelihood of having reduced symptomology associated with a neuropsychiatric disorder than an individual having a “C” at that position.

8. A method according to claim 7, wherein the neuropsychiatric disorder is schizophrenia.

9. A method for predicting the likelihood that an individual will have reduced symptomology associated with a neuropsychiatric disorder, comprising the steps of:
a) obtaining a DNA sample from an individual to be assessed; and
b) determining the nucleotide present at the polymorphic position in SEQ ID NO: 2,
wherein the presence of an “G” at this position indicates that the individual has a greater likelihood of having reduced symptomology associated with a neuropsychiatric disorder than an individual having a “A” at that position.

10. A method according to claim 10, wherein the neuropsychiatric disorder is schizophrenia.

11. A method of diagnosing or aiding in the diagnosis of a neuropsychiatric disorder in an individual comprising
a) obtaining a nucleic acid sample from the individual; and
b) determining the nucleotide present at one or more of the polymorphic positions in SEQ ID NO: 1 and in SEQ ID NO: 2,
wherein presence of one or more of a C at the polymorphic position in SEQ ID NO: 1, or an A at the polymorphic position in SEQ ID NO: 2 is indicative of increased likelihood of a neuropsychiatric disorder in the individual as compared with an individual having one or more of a T at the polymorphic position in SEQ ID NO: 1, or a G at the polymorphic position in SEQ ID NO: 2.

12. The method of claim 11, wherein the nucleotides present at the polymorphic positions in both SEQ ID NO: 1 and SEQ ID NO: 2 are determined.

13. The method of claim 11, wherein the neuropsychiatric disorder is schizophrenia.

14. A method of diagnosing or aiding in the diagnosis of a neuropsychiatric disorder in an individual comprising
a) obtaining a nucleic acid sample from the individual; and
b) determining the nucleotide present at one or more of the polymorphic positions in SEQ ID NO: 1 and in SEQ ID NO: 2,
wherein presence of one or more of a T at the polymorphic position in SEQ ID NO: 1, or a G at the polymorphic position in SEQ ID NO: 2 is indicative of decreased likelihood of a neuropsychiatric disorder in the individual as compared with an individual having one or more of a C at the polymorphic position in SEQ ID NO: 1, or an A at the polymorphic position in SEQ ID NO: 2.

15. The method of claim 14, wherein the nucleotides present at the polymorphic positions in both SEQ ID NO: 1 and SEQ ID NO: 1 are determined.

16. The method of claim 14, wherein the neuropsychiatric disorder is schizophrenia.

17. A method for predicting the likelihood that an individual will have a neuropsychiatric disorder, comprising the steps of:
a) obtaining a DNA sample from an individual to be assessed; and
b) determining the nucleotide present at one or more of the polymorphic positions in SEQ ID NO: 1 and in SEQ ID NO: 2,
wherein presence of one or more of a C at the polymorphic position in SEQ ID NO: 1, or an A at the polymorphic position in SEQ ID NO: 2 is indicative of increased likelihood of a neuropsychiatric disorder in the individual as compared with an individual having one or more of a T at the polymorphic position in SEQ ID NO: 1, or a G at the polymorphic position in SEQ ID NO: 2.

18. The method according to claim 17, wherein the nucleotides present at the polymorphic positions in both SEQ ID NO: 1 and SEQ ID NO: 2 are determined.

19. The method according to claim 17, wherein the individual is an individual at risk for development of a neuropsychiatric disorder.

20. The method according to claim 17, wherein the neuropsychiatric disorder is schizophrenia.

21. A method for predicting the likelihood that an individual will have reduced symptomology associated with a neuropsychiatric disorder, comprising the steps of:
a) obtaining a DNA sample from an individual to be assessed; and
b) determining the nucleotide present at one or more of the polymorphic positions in SEQ ID NO: 1 and in SEQ ID NO: 2,
wherein presence of one or more of a T at the polymorphic position in SEQ ID NO: 1, or a G at the polymorphic position in SEQ ID NO: 2 is indicative of decreased likelihood of a neuropsychiatric disorder in the individual as compared with an individual having one or more of a C at the polymorphic position in SEQ ID NO: 1, or an A at the polymorphic position in SEQ ID NO: 2.

22. The method according to claim 21, wherein the nucleotides present at the polymorphic positions in both SEQ ID NO: 1 and SEQ ID NO: 2 are determined.

23. The method according to claim 21, wherein the individual is an individual at risk for development of a neuropsychiatric disorder.

24. The method according to claim 21, wherein the neuropsychiatric disorder is schizophrenia.

25. An oligonucleotide microarray having immobilized thereon a plurality of probes, wherein at least one of said probes is specific for the variant form of the single nucleotide polymorphism in SEQ ID NO: 1.

26. An oligonucleotide microarray having immobilized thereon a plurality of probes, wherein at least one of said probes is specific for the reference form of the single nucleotide polymorphism in SEQ ID NO: 1.

27. An oligonucleotide microarray having immobilized thereon a plurality of probes, wherein at least one of said probes is specific for the variant form of the single nucleotide polymorphism in SEQ ID NO: 2.

28. An oligonucleotide microarray having immobilized thereon a plurality of probes, wherein at least one of said probes is specific for the reference form of the single nucleotide polymorphism in SEQ ID NO: 2.

29. An oligonucleotide microarray having immobilized thereon a plurality of probes, wherein at least one of said probes is specific for the variant form of the single nucleotide polymorphism in SEQ ID NO: 1 and wherein at least one of said probes is specific for the variant form of the single nucleotide polymorphism in SEQ ID NO: 2.

30. An oligonucleotide microarray having immobilized thereon a plurality of probes, wherein at least one of said probes is specific for the reference form of the single nucleotide polymorphism in SEQ ID NO: 1 wherein at least one of said probes is specific for the reference form of the single nucleotide polymorphism in SEQ ID NO: 2.
